(19) United States
(12) Patent Application Publication
(10) Pub. No.: US 2020/0368536 A1
(43) Pub. Date: Nov. 26, 2020

(54) METHODS AND SYSTEMS FOR LABELING ARRHYTHMIAS BASED ON HEART SOUNDS

(71) Applicant: PACESETTER, INC., Sytlmar, CA (US)

(72) Inventors: Jong GILL, Valencia, CA (US); Gene A. BORNZIN, Santa Monca, CA (US)

(21) Appl. No.: 16/417,194

(22) Filed: May 20, 2019

(51) Int. Cl. A61N 1/365; A61B 5/0464; A61B 5/0472; A61B 5/11; A61B 5/00; A61N 1/362

(52) U.S. Cl. CPC A61N 1/36585; A61B 5/0464; A61B 5/0472; A61B 2562/0219; A61B 5/7221; A61N 1/3621; A61B 5/1118

(57) ABSTRACT

A computer implemented method and system for labeling types of heart arrhythmias based on cardiac activity are provided. The method is under control of one or more processors of an implantable medical device (IMD) configured with specific executable instruction. The method obtains cardiac activity (CA) signals at electrodes of the IMD during cardiac beats, declares a ventricular tachycardia (VT) episode based on the CA signals and obtains acceleration signatures, at an accelerometer of the IMD, indicative of heart sounds generated during the cardiac beats. The method analyzes an S1 characteristic of interest (COI) from the acceleration signature to identify the VT episode as a stable or non-stable VT episode and labels the VT episode as stable or non-stable based on the analyzing operation.

### BACKGROUND

Embodiments herein generally relate to methods and systems for labeling types of arrythmias, and more particularly, to methods and systems that utilize heart sounds in connection with labeling types of arrhythmias.

Implantable medical devices (IMD) are well known in the art. The IMD may take the form of implantable defibrillators or cardioverters which treat accelerated rhythms of the heart such as fibrillation. The IMD may also take the form of implantable pacemakers which maintains the heart rate above a prescribed limit, such as, for example, to treat a bradycardia. Implantable medical devices may also incorporate more than one of a pacemaker, a cardioverter and a defibrillator. Defibrillators may include “shock only” functionality or, in addition to shocking functionality, a defibrillator may be capable of providing cardiac resynchronization therapy (CRT) functionality.

IMDs are coupled to one or more leads that include electrodes to sense one or more types of information and to deliver various types of therapy. The IMDs typically include various sensing circuitry and logic that monitor a heart for cardiac signals, and analyze the cardiac signals to identify normal sinus rhythm, arrhythmias, and the like. IMDs are configured to deliver therapies based on the identification of arrhythmias and the like. However, IMDs may incorrectly interpret the cardiac activity signals and declare a false arrhythmia due to sensing issues or hardware dysfunctions. Additionally, or alternatively, IMDs may correctly interpret the cardiac signals and declare a true arrhythmia, but while the patient is still conscious because the arrhythmia is well-tolerated. Therapies and/or interventions delivered by IMDs with defibrillation functionality are painful. When defibrillation therapies and/or interventions are unnecessarily administered, they have a deleterious impact on quality of life. Unnecessary administration of defibrillation therapies and/or interventions occur at a rate that renders them a concern in the long-term management of the corresponding patient population. Accordingly, a desire remains to improve the reliability of arrhythmia detection and differentiation, and to reduce the unnecessary administration of defibrillation therapies and/or interventions.

### SUMMARY

In accordance with embodiments herein, a computer implemented method for labeling types of heart arrhythmias based on cardiac activity is provided. The method is under control of one or more processors of an implantable medical device (IMD) configured with specific executable instruction. The method obtains cardiac activity (CA) signals at electrodes of the IMD during cardiac beats, declares a ventricular tachycardia (VT) episode based on the CA signals and obtains acceleration signatures, at an accelerometer of the IMD, indicative of heart sounds generated during the cardiac beats. The method analyzes an S1 characteristic of interest (COI) from the acceleration signature to identify the VT episode as a stable or non-stable VT episode and labels the VT episode as stable or non-stable based on the analyzing operation.

Optionally, the declaring may include declaring the VT episode as a candidate VT episode. The method may further comprise analyzing S1 and S2 heart sound (HS) components of the acceleration signatures to confirm or deny the candidate VT episode. The analyzing the S1 and S2 HS components may comprise at least one of i) comparing amplitudes of the S1 and S2 HS components to a predetermined threshold ii) comparing the amplitudes of the S1 and S2 HS components to a dynamic threshold that is derived based on S1 and S2 trends over a previous window of the cardiac beats; and iii) comparing the amplitudes of the S1 and S2 HS components to S1 and S2 amplitudes that were acquired previously during an exercise activity and while a heart rate was in a range corresponding to a current heart rate associated with the VT episode.

Optionally, the declaring may include declaring the VT episode as a candidate VT episode. The analyzing may further comprise analyzing an amplitude of an S1 heart sound component to confirm or deny the candidate VT episode. The S1 COI may be S1 amplitude. The analyzing may comprise tracking a level of the S1 amplitude during or after the VT episode, labeling the VT episode as stable when the level of the S1 amplitude remains above an amplitude level threshold for a select portion of the VT episode and labeling the VT episode as non-stable when the level in the S1 amplitude falls below the amplitude level threshold for the select portion of the VT episode.

Optionally, the S1 COI may be S amplitude variation. The analyzing may comprise tracking variation in the S1 amplitude during or after the VT episode, labeling the VT episode as stable when the variation of the S1 amplitude is less than an amplitude variation threshold for a select portion of the VT episode and labeling the VT episode as non-stable when the variation in the S1 amplitude exceeds the amplitude variation threshold for the select portion of the VT episode. The S1 COI may be S1 amplitude level and variation. The analyzing may comprise tracking variation and a level of the S1 amplitude during or after the VT episode and labeling the VT episode as stable or non-stable based on a relation between the variation and level of the S1 amplitude and variation and amplitude thresholds.

Optionally, the method may identify over a series of the cardiac beats, a QRS to S1 Interval corresponding to a period of time between i) a feature of interest in a QRS complex of the corresponding cardiac beats and ii) a feature of interest in the S1 heart sound. The method may determine a variability of the QRS to S1 interval over the series of the cardiac beats. The labeling may comprise labeling the VT episode as stable or non-stable based on the variability of the QRS to S1 interval. The method may identify, over a series of the cardiac beats, a QRS complex from the corresponding cardiac beats. The method may detect skipped S1 heart sounds that do not occur within an S search window following a corresponding QRS complex. The labeling may comprise labeling the VT episode as stable or non-stable based on the skipped S1 heart sounds.

Optionally, the method may analyze the acceleration signature for an activity level and may confirm or deny the VT episode based on a relation between the activity level and a threshold. The activity level may be indicative of a non-VT episode when the activity level exceeds the threshold. The activity level may be indicative of the VT episode when the activity level falls below the threshold. The method may deliver a first therapy when the VT episode is labeled as stable and may deliver a second therapy when the VT episode is labeled as non-stable.

In accordance with embodiments herein, a system for labeling types of heart arrhythmias based on cardiac activity is provided. The system includes one or more processors. A memory is coupled to the one or more processors. The memory stores program instructions. The program instructions are executable by the one or more processors to obtain cardiac activity (CA) signals at electrodes of the IMD during cardiac beats, declare a ventricular tachycardia (VT) episode based on the CA signals and obtain acceleration signatures, at an accelerometer of the IMD, indicative of heart sounds generated during the cardiac beats. The system analyzes an S1 characteristic of interest (COI) from the acceleration signature to identify the VT episode as a stable or non-stable VT episode and labels the VT episode as stable or non-stable based on the analyzing operation.

Optionally, the declaring may include declaring the VT episode as a candidate VT episode. The program instructions may be further executable by the one or more processors to analyze S1 and S2 HS components of the acceleration signatures to confirm or deny the candidate VT episode. The analyze S1 and S2 HS components may comprise at least one of i) compare amplitudes of the S1 and S2 heart sound (HS) components to a predetermined threshold, ii) compare the amplitudes of the S1 and S2 HS components to a dynamic threshold that is derived based on S1 and S2 trends over a previous window of the cardiac beats and iii) compare the amplitudes of the S1 and S2 HS components to S1 and S2 amplitudes that were acquired previously during an exercise activity and while a heart rate was in a range corresponding to a current heart rate associated with the VT episode.

Optionally, the declaring may include declaring the VT episode as a candidate VT episode. The analyzing may further comprise analyzing an amplitude of an S1 HS component to confirm or deny the candidate VT episode. The S1 COI may be S1 amplitude. The analyze may further comprise tracking a level of the S1 amplitude during or after the VT episode. The system may label the VT episode as stable when the level of the S1 amplitude remains above an amplitude level threshold for a select portion of the VT episode and may label the VT episode as non-stable when the level in the S1 amplitude falls below the amplitude level threshold for the select portion of the VT episode.

Optionally, the S1 COI may be S1 amplitude variation. The analyzing may comprise tracking variation in the S1 amplitude during or after the VT episode, labeling the VT episode as stable when the variation of the S1 amplitude is less than an amplitude variation threshold for a select portion of the VT episode and labeling the VT episode as non-stable when the variation in the S1 amplitude exceeds the amplitude variation threshold for the select portion of the VT episode. The S1 COI may be S1 amplitude level and variation. The analyzing may comprise tracking variation and a level of the S1 amplitude during or after the VT episode and labeling the VT episode as stable or non-stable based on a relation between the variation and level of the S1 amplitude and variation and amplitude thresholds.

Optionally, the program instructions may be further executable by the one or more processors to identify, over a series of the cardiac beats, a QRS to S1 interval corresponding to a period of time between i) a feature of interest in a QRS complex of the corresponding cardiac beats and ii) a feature of interest in the S1 heart sound and may determine a variability of the QRS to S1 interval over the series of the cardiac beats. The labeling may comprise labeling the VT episode as stable or non-stable based on the variability of the QRS to S1 interval. The program instructions may be further executable by the one or more processors to identify, over a series of the cardiac beats, a QRS complex from the corresponding cardiac beats and may detect skipped S1 heart sounds that do not occur within an S1 search window following a corresponding QRS complex. The labeling may comprise labeling the VT episode as stable or non-stable based on the skipped S1 heart sounds.

### DESCRIPTION OF THE DRAWINGS

FIG. 1 illustrates a graphical representation of a heart with an implantable medical device (IMD) for providing defibrillation and optionally other therapy in accordance with embodiments herein.

FIG. 2 illustrates a block diagram of the IMD formed in accordance with embodiments herein.

FIG. 3 illustrates force vectors experienced by the ICM in accordance with embodiments herein.

FIG. 4 illustrates a flow block diagram of a method of labeling types of heart arrythmias based on cardiac activity.

FIG. 5 is a graph illustrating examples of activity levels in connection with different types of cardiac activity in accordance with embodiments herein.

FIG. 6 is a graph illustrating examples of S1 amplitude levels in connection with different types of cardiac activity in accordance with embodiments herein.

FIG. 7 is a graph illustrating examples of S1 amplitude variability in connection with different types of cardiac activity in accordance with embodiments herein.

FIG. 8 is a graph illustrating examples of S1 amplitude variability versus S1 amplitude level in connection with different types of cardiac activity in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

### Terms

The terms “state”, “patient state”, and “activity level” refer to types of activity currently experienced by a patient, including a stationary state, rest state, exercise state, walking state, and the like.

The term “IMD location” refers collectively to both: i) a position of an IMD, with respect to a reference position, and ii) an orientation of the IMD with respect to a reference orientation. The IMD location may be continuously or periodically determined relative to gravitational force. The reference position and orientation may be defined, during a calibration operation, relative to a global coordinate system. As a patient moves and/or shift postures, the IMD within the patient similarly translationally moves along one or more linear axis (e.g., X, Y and Z directions) from the reference position. Additionally, or alternatively, as a patient moves and/or shift postures, the IMD within the patient similarly rotationally moves along one or more rotational axis (e.g., pitch, yaw and roll directions) from the reference orientation.

The terms “cardiac activity signal”, “cardiac activity signals”, “CA signal” and “CA signals” (collectively “CA signals”) are used interchangeably throughout to refer to an analog or digital electrical signal recorded by two or more electrodes positioned subcutaneous or cutaneous, where the electrical signals are indicative of cardiac electrical activity. The cardiac activity may be normal/healthy or abnormal/arrhythmic. Non-limiting examples of CA signals include ECG signals collected by cutaneous electrodes, and EGM signals collected by subcutaneous electrodes and/or by electrodes positioned within or proximate to the heart wall and/or chambers of the heart.

The phrases “arrhythmia treatment”, “in connection with treating a heart condition” and similar phrases, as used herein include, but are not limited to, delivering an electrical stimulation or drug therapy to a heart condition. By way of example, treating a heart condition may include, in whole or in part, i) identifying a progression of heart failure over time; ii) confirming an arrhythmia identified by an arrhythmia detection process; iii) Instructing the patient to perform a posture recalibration procedure and/or iv) delivering a therapy.

The terms “beat” and “cardiac event” are used interchangeably and refer to both normal and/or abnormal events.

The terms “normal” and “sinus” are used to refer to events, features, and characteristics of, or appropriate to, a heart's healthy or normal functioning.

The terms “abnormal,” or “arrhythmic” are used to refer to events, features, and characteristics of, or appropriate to, an unhealthy or abnormal functioning of the heart.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like.

The terms “device shift” and “IMD shift,” as used herein, refer to a change In position and/or orientation of an IMD within a subcutaneous implant region. By way of example, an IMD drift may occur when an IMD moves in one or more of six degrees of freedom within the subcutaneous implant region. As a further example, a reference point and/or longitudinal axis of an IMD may move In an X, Y and/or Z direction and/or rotate in a pitch, yaw and/or tilt direction with respect to a reference point in a patient (e.g., a reference point on the heart).

The term “obtains” and “obtaining”, as used in connection with data, signals, information and the like, include at least one of i) accessing memory of an external device or remote server where the data, signals, information, etc. are stored, ii) receiving the data, signals, information, etc. over a wireless communications link between the ICM and a local external device, and/or iii) receiving the data, signals, information, etc. at a remote server over a network connection. The obtaining operation, when from the perspective of an ICM, may include sensing new signals In real time, and/or accessing memory to read stored data, signals, information, etc. from memory within the ICM. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer,

### System for Labeling Arrythmias

In accordance with embodiments herein, methods and systems are provided for labeling types of arrythmias. The types of arrythmias may be labeled based on confirming heart arrythmias initially declared based on cardiac activity and, optionally, differentiating heart arrythmias based on cardiac activity and accelerometer signatures detected and retrieved by an IMD. Embodiments herein increase the quality of life and improve the long-term management of relevant patient populations by managing (e.g., reducing or eliminating) unnecessary administration of defibrillation therapies and/or interventions.

In accordance with embodiments herein, the IMD includes an accelerometer to collect accelerometer signatures indicative of activity level of a patient and heart sounds of the corresponding cardiac beats. The accelerometer may provide data or accelerometer signatures indicative of patent activity and heart sounds. The accelerometer signatures indicative of patient activity may be used to determine whether the patient is active during the time an arrhythmia episode is declared by the device based on CA signals. In one example, if the arrhythmia episode is appropriate, the patient is likely to have a resting state, corresponding to unconsciousness. In another example, it is possible for the patient to be active (e.g., exhibit some level of activity) when the diagnosis is inappropriate due to, for example and without limitation, oversensing of T-waves and the like. Additionally, or alternatively, the accelerometer signatures indicative of heart sounds may provide data indicative of whether the arrhythmia episode is stable or unstable. HS features related to cardiac valve(s) closing, such as S1, S2, and the like, may be used to determine whether the arrhythmia episode is stable and tolerable, in which case a perfusion to the brain is still maintained.

Accordingly, in one example, using accelerometer signatures indicative of patient activity in conjunction with cardiac signals enables confirmation or denial of an arrhythmia diagnosis. For example, if the activity level corresponds to an active state, and thus a conscious patient, that may indicate a true arrhythmia is well tolerated and does not need administration of a treatment. Conversely, if the activity level of the patient corresponds to an inactive patient or a patient exhibiting a rapid decrease in activity level, the arrhythmia diagnosis is confirmed. In another example, if the activity level corresponds to an active state, the arrhythmia diagnosis may be due to sensing issues or hardware dysfunction. In accordance with additional or alternative embodiments herein, the accelerometer signatures indicative of heart sounds enables confirmation or denial of an arrhythmia diagnosis and/or differentiation between stable and non-stable arrythmias.

Embodiments may be implemented in connection with one or more implantable medical devices (IMDs), including IMDs that include an ICD functionality. Non-limiting examples of IMDs include one or more of neurostimulator devices, implantable cardiac monitoring and/or therapy devices. For example, the IMD may represent a cardiac monitoring device, pacemaker, cardioverter, cardiac rhythm management device, defibrillator, neurostimulator, leadless monitoring device, leadless pacemaker, an external shocking device (e.g., an external wearable defibrillator), and the like. For example, the IMD may be a subcutaneous IMD that includes one or more structural and/or functional aspects of the device(s) described in U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties. Additionally, or alternatively, the IMD may include one or more structural and/or functional aspects of the device(s) described in U.S. Pat. No. 9,333,351 “Neurostimulation Method And System To Treat Apnea” and U.S. Pat. No. 9,044,610 “System And Methods For Providing A Distributed Virtual Stimulation Cathode For Use With An Implantable Neurostimulation System”, which are hereby incorporated by reference. Further, one or more combinations of IMDs may be utilized from the above incorporated patents and applications in accordance with embodiments herein.

Additionally, or alternatively, the IMD may include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method Including The Same”, which are hereby incorporated by reference. Additionally, or alternatively, the IMD may include one or more structural and/or functional aspects of the device(s) described in U.S. Pat. No. 8,391,980 “Method And System For Identifying A Potential Lead Failure In An Implantable Medical Device”, U.S. Pat. No. 9,232,485 “System And Method For Selectively Communicating With An Implantable Medical Device”, EP Application No. 0060404 “Defibrillator” and, U.S. Pat. No. 5,334,045 “Universal Cable Connector For Temporarily Connecting Implantable Leads And Implantable Medical Devices With A Non-Implantable System Analyzer”, which are hereby incorporated by reference.

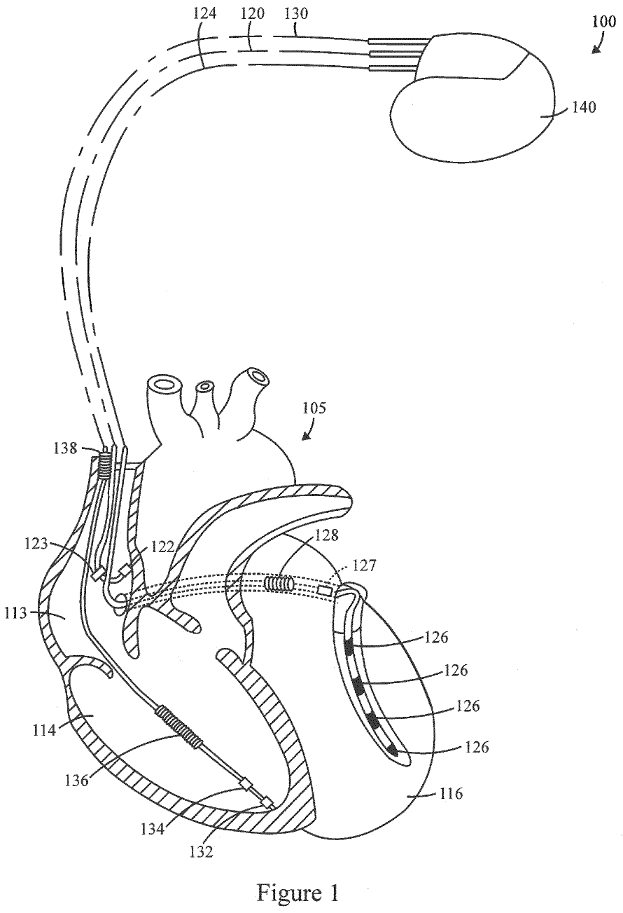

FIG. 1 illustrates an IMD 100 in electrical communication with multiple leads implanted into a patient's heart 105 for delivering multi-chamber stimulation and sensing cardiac activity according to an embodiment. The IMD 100 may be a dual-chamber stimulation device, including an IMD, capable of treating both fast and slow arrhythmias with stimulation therapy, including cardioversion, defibrillation, and pacing stimulation, including cardiac resynchronization therapy (CRT). Optionally, the IMD 100 may be configured for single site or multi-site left ventricular (MSLV) pacing, which provides pacing pulses at more than one site within the LV chamber each pacing cycle. The IMD 100 may be referred to herein as IMD 100. To provide atrial chamber pacing stimulation and sensing, IMD 100 is shown in electrical communication with a heart 105 by way of a left atrial (LA) lead 120 having an atrial tip electrode 122 and an atrial ring electrode 123 implanted in the atrial appendage 113. IMD 100 is also in electrical communication with the heart 105 by way of a right ventricular (RV) lead 130 having, in this embodiment, a ventricular tip electrode 132, an RV ring electrode 134, an RV coil electrode 136, and a superior vena cava (SVC) coil electrode 138. The RV lead 130 is transvenously inserted into the heart 105 so as to place the RV coil electrode 136 in the RV apex, and the SVC coil electrode 138 in the superior vena cava. Accordingly, the RV lead 130 is capable of receiving cardiac signals and delivering stimulation in the form of pacing and shock therapy to the right ventricle 114 (also referred to as the RV chamber).

To sense left atrial and ventricular cardiac signals and to provide left ventricle 116 (e.g., left chamber) pacing therapy, IMD 100 is coupled to an LV lead 124 designed for placement in various locations such as the “CS region”, the epicardial space, etc. As used herein, the phrase “CS region” refers to the venous vasculature of the left ventricle, including any portion of the coronary sinus (CS), great cardiac vein, left marginal vein, left posterior ventricular vein, middle cardiac vein, and/or small cardiac vein or any other cardiac vein accessible by the coronary sinus. In an embodiment, an LV lead 124 is designed to receive atrial and ventricular cardiac signals and to deliver one or more of left ventricular pacing therapy using LV electrodes 126, left atrial pacing therapy using at least an LA ring electrode 127, and shocking therapy using at least an LA coil electrode 128. Although three leads 120, 124, and 130 are shown in FIG. 1, fewer or additional leads with various numbers of pacing, sensing, and/or shocking electrodes may optionally be used. For example, the LV lead 124 may have more or less than four LV electrodes 126.

When selecting a target venous branch for the LV lead 124, several factors may be taken into account. For example, it may be desirable to maximize the LV mass that may be captured by the LV lead 124. Accordingly, to maximize LV mass exposure, certain venous branches may be preferred for positioning the LV lead 124. Further, a diameter and trajectory of the venous branch is also considered to ensure that the venous branch will support chronic stability of an LV lead 124. Passive fixation of the LV lead 124 may be established through the anatomy of the host venous branch which causes the LV lead 124 to extend the distal portion thereof in a manner that differs from the LV lead's preformed shape. Optionally, additional factors to be considered when placing the LV lead 124 may include reducing myocardial capture thresholds, avoiding atrial and phrenic nerve stimulation and the like.

The LV electrodes 126 are configured such that each electrode may be utilized to deliver pacing pulses and/or sense pacing pulses (e.g., monitor the response of the LV tissue to a pacing pulse). In a pacing vector or a sensing vector, each LV electrode 126 may be controlled to function as a cathode (negative electrode). Pacing pulses may be directionally provided between electrodes to define a pacing vector. In a pacing vector, a generated pulse is applied to the surrounding myocardial tissue through the cathode. The electrodes that define the pacing vectors may be electrodes in the heart 105 or located externally to the heart 105 (e.g., on a housing/case device 140). For example, the housing/case 140 may be referred to as the CAN 140 and function as an anode in unipolar pacing and/or sensing vectors. The RV coil 136 may also function as an anode in unipolar pacing and/or sensing vectors. The LV electrodes 126 may be used to provide various different vectors. Some of the vectors are intraventricular LV vectors (e.g., vectors between two of the LV electrodes 126), while other vectors are interventricular vectors (e.g., vectors between an LV electrode 126 and the RV coil 136 or another electrode remote from the left ventricle 116).

It is recognized that various other types of leads and IMDs may be used with various other types of electrodes and combinations of electrodes. The foregoing electrode types/combinations are provided as non-limiting examples.

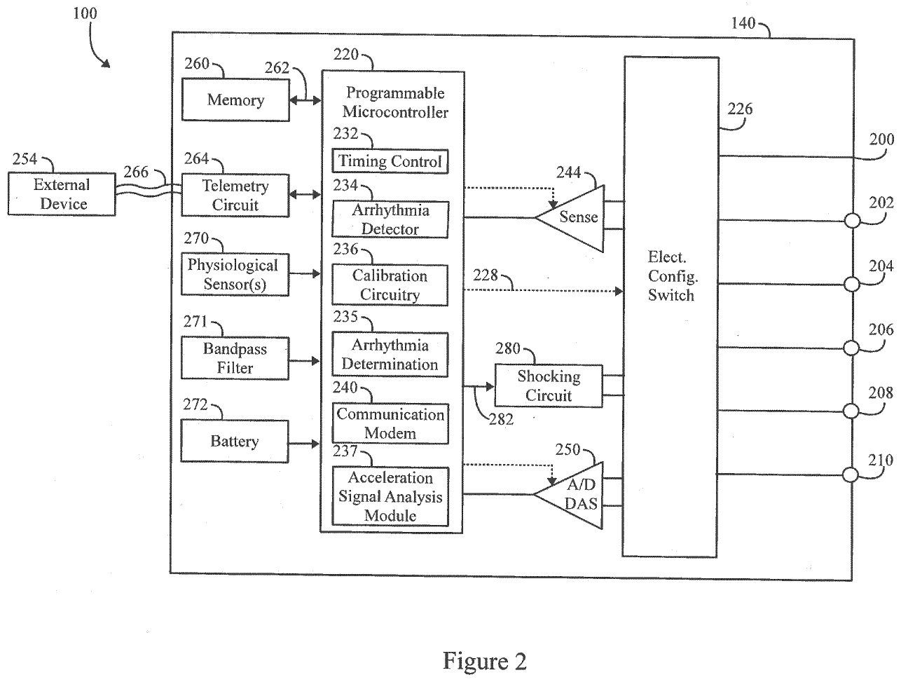

FIG. 2 shows an example block diagram of the IMD 100 formed in accordance with embodiments herein. The IMD 100 may treat both fast and slow arrhythmias with stimulation therapy, including cardioversion, pacing stimulation, an implantable cardioverter defibrillator, suspend tachycardia detection, tachyarrhythmia therapy, and/or the like.

The IMD 100 has a housing 140 to hold the electronic/computing components. The housing 140 (which is often referred to as the “can,” “case,” “encasing,” or “case electrode”) may be programmably selected to act as an electrode for certain sensing modes. Housing 140 further includes a connector (not shown) with at least one terminal 200 and optionally additional terminals 202, 204, 206, 208, 210. The terminals 200, 202, 204, 206, 208, 210 may be coupled to sensing electrodes that are provided upon or immediately adjacent the housing 140. Optionally, more or less than six terminals 200, 202, 204, 206, 208, 210 may be provided in order to support more or less than six sensing electrodes. Additionally, or alternatively, the terminals 200, 202, 204, 206, 208, 210 may be connected to one or more leads having one or more electrodes provided thereon, where the electrodes are located in various locations about the heart. The type and location of each electrode may vary.

The IMD 100 includes a programmable microcontroller 220 that controls various operations of the IMD 100, including cardiac monitoring. Microcontroller 220 includes a microprocessor (or equivalent control circuitry), RAM and/or ROM memory, logic and timing circuitry 704, state machine circuitry, and I/O circuitry. Microcontroller 220 includes an arrhythmia detector 234 that is configured to analyze the cardiac activity signals to identify the existence of an arrhythmia. The microcontroller also includes arrhythmia determination circuitry 235 for analyzing the CA signals to assess a presence or absence of R-waves within the cardiac beats, declaring a candidate arrhythmia based on the absence of at least one R-wave from the cardiac beats, and evaluating the acceleration signatures for ventricular events (VEs) and activity levels to confirm or deny the candidate arrhythmia. In particular, the arrhythmia determination circuitry 235 analyzes the CA signals to determine a candidate arrhythmia, and utilizes the acceleration signatures of VEs and activity level to verify the determination. Consequently, arrhythmia detection accuracy is increased. Also, the microcontroller 220 further controls a shocking circuit 280 by way of a control signal 282. The shocking circuit 280 generates shocking pulses that are applied to the heart of the patient through at least two shocking electrodes. The shocking pulses may be selected from the LA coil electrode 128, the RV coil electrode 136, and/or the SVC coil electrode 138 (all three electrodes shown in FIG. 1). The CAN 140 may act as an active electrode in combination with the RV coil electrode 136, or as part of a split electrical vector using the SVC coil electrode 138 or the LA coil electrode 128 (e.g., with the RV coil electrode 136 as a common electrode).

The microcontroller 220 may also include an acceleration signature analysis (ASA) module 237 configured to implement one or more of the operations discussed herein. The ASA module is configured to be a computer implemented method for detecting arrhythmias in cardiac activity. The ASA module obtains cardiac activity (CA) signals, at the electrodes of an IMD, in connection with multiple cardiac beats and in connection with different IMD locations relative to gravitational force. The method obtains acceleration signatures, at a sensor of the IMD, indicative of heart sounds generated during the cardiac beats and obtains device location information, at the IMD, with respect to the gravitational force during the cardiac beats indicative of patient activity. The ASA processor 237 evaluates the acceleration signatures for activity level and characteristics of ventricular events (VEs) to confirm or deny a candidate arrhythmia episode (e.g., a candidate VT episode) determined by the arrhythmia determination circuitry 235.

The microcontroller 220 may also include calibration circuitry 236 that obtains calibration acceleration signatures at an accelerometer, or physiological sensor 270 that is indicative of heart sounds generated in connection with activity levels of a patient. The postures may include supine, laying on a right side, laying on are left side, angled, or the like. In one example, the acceleration signatures are indicative of heart sounds generated in connection with first and second postures of a patient. After the calibration procedure, the calibration circuitry 236 utilizes the calibration acceleration signatures to determine an axis of the accelerometer associated with a current posture. The confirmation acceleration signatures are obtained along the axis of the accelerometer In connection with the analyzing the far field CA signals.

Although not shown, the microcontroller 220 may further include other dedicated circuitry and/or firmware/software components that assist in monitoring various conditions of the patient's heart and managing pacing therapies.

A switch 226 is optionally provided to allow selection of different electrode configurations under the control of the microcontroller 220. The electrode configuration switch 226 may include multiple switches for connecting the desired electrodes to the appropriate I/O circuits, thereby facilitating electrode programmability. The switch 226 is controlled by a control signal 228 from the microcontroller 220. Optionally, the switch 226 may be omitted and the I/O circuits directly connected to a housing electrode via terminal 200 and one or more other electrodes via terminals 202, 204, 206, 208, 210.

The IMD 100 is further equipped with a communication modem (modulator/demodulator) 240 to enable wireless communication. In one implementation, the communication modem 240 uses high frequency modulation, for example using RF, Bluetooth or Bluetooth Low Energy telemetry protocols. The signals are transmitted in a high frequency range and will travel through the body tissue in fluids without stimulating the heart or being felt by the patient. The communication modem 240 may be implemented in hardware as part of the microcontroller 220, or as software/firmware instructions programmed into and executed by the microcontroller 220. Alternatively, the modem 240 may reside separately from the microcontroller as a standalone component. The modem 240 facilitates data retrieval from a remote monitoring network. The modem 240 enables timely and accurate data transfer directly from the patient to an electronic device utilized by a physician.

The IMD 100 includes sensing circuit 244 selectively coupled to one or more electrodes that perform sensing operations, through the switch 226 to detect cardiac activity data indicative of cardiac activity. The sensing circuit 244 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 226 may be used to determine the sensing polarity of the cardiac signal by selectively closing the appropriate switches.

In the example of FIG. 2, a single sensing circuit 244 is illustrated. Optionally, the IMD 100 may include multiple sensing circuits, similar to sensing circuit 244, where each sensing circuit is coupled to two or more electrodes and controlled by the microcontroller 220 to sense electrical activity detected at the corresponding two or more electrodes. The sensing circuit 244 may operate in a unipolar sensing configuration or a bipolar sensing configuration. Optionally, the sensing circuit 244 may be removed entirely, and the microcontroller 220 perform the operations described herein based upon the CA signals from the A/D data acquisition system 250 directly coupled to the electrodes. The output of the sensing circuit 244 is connected to the microcontroller 220 which, in turn, determines when to store the cardiac activity data of CA signals (digitized by the A/D data acquisition system 250) in the memory 260. The CA signals are analyzed to determine a candidate arrhythmia that may be verified by analysis of acceleration signatures as described herein.

The IMD 100 further includes an analog-to-digital A/D data acquisition system (DAS) 250 coupled to one or more electrodes via the switch 226 to sample cardiac activity signals across any pair of desired electrodes. The ASA processer 237 may be applied to signals from the sensing circuit 244 and/or the DAS 250.

By way of example, the external device 254 may represent a bedside monitor installed in a patient's home and utilized to communicate with the IMD 100 while the patient is at home, in bed or asleep. The external device 254 may be a programmer used in the clinic to interrogate the IMD 100, retrieve data and program detection criteria and other features. The external device 254 may be a handheld device (e.g., smartphone, tablet device, laptop computer, smartwatch and the like) that may be coupled over a network (e.g., the Internet) to a remote monitoring service, medical network and the like. The external device 254 may communicate with a telemetry circuit 264 of the IMD through a communication link 266. The external device 254 facilitates access by physicians to patient data as well as permitting the physician to review real-time CA signals while collected by the IMD 100.

The microcontroller 220 is coupled to a memory 260 by a suitable data/address bus 262. The memory 260 stores the acceleration signatures, reference posture data sets, cardiac activity signals, as well as the markers and other data content associated with detection and determination of the arrhythmia.

The IMD 100 may further include one or more physiologic sensors 270. For example, the physiologic sensor 270 may represent one or more accelerometers, such as a three-dimensional (3D) accelerometer. The sensor 270 may utilize a piezoelectric, a piezoresistive, and/or capacitive components are commonly used to convert the mechanical motion of the 3D accelerometer into an electrical signal received by the microcontroller 220. By way of example, the 3-D accelerometer may generate three electrical signals indicative of motion in three corresponding directions, namely X, Y and Z directions. The electrical signals associated with each of the three directional components may be divided into different frequency components to obtain different types of information therefrom.

The physiologic sensor 270 collects device location information with respect to gravitational force while the IMD collects cardiac activity signals in connection with multiple cardiac beats. The microcontroller 220 may utilize the signals from the physiologic sensor 270 in the manner described in U.S. Pat. No. 6,937,900, titled “AC/DC Multi-Axis Accelerometer For Determining A Patient Activity And Body Position,” the complete subject matter which is expressly incorporated herein by reference. While shown as being included within the housing 140, the physiologic sensor(s) 270 may be external to the housing 140, yet still, be implanted within or carried by the patient.

The physiologic sensor 270 may be further configured to obtain acceleration signatures indicative of heart sounds generated during cardiac beats. The acceleration signatures from the sensor 270 are provided to the microcontroller 220 and are analyzed by the acceleration signature analysis process 237. In one example of the sensor 270 varies readings as a result of positioning of the patient. For instance, if the IMD is placed on a flat surface, the z-axis of the accelerometer indicates 1 g (gravity) while the x and y axis gravity data are zero because each axis is perpendicular to gravity. If the patient changes position, a new position is represented by a unique combination of x, y, z values with respect to gravity. As such, the position of the IMD is indicative of gravity influencing 3-axis of the accelerometer that is unique to a particular position and is not the result of heart sound.

In one example, the accelerator signatures may be an AC-high frequency component from the 3-D accelerometer. The AC-high frequency component may correspond to one or more axes of the accelerometer and, additionally, or alternatively, may represent a composite AC-high frequency component formed from a combination (e.g., a sum) of the AC-high frequency components from the three electrical signals. The composite AC-high frequency component generally represents the acceleration signature that is indicative of heart sounds produced during a corresponding cardiac cycle. The AC-high frequency component may include signals having a frequency of 10 KHz or more, and more preferably in the range of 10-100 kHz.

In yet another example, the three directional signals generated by the 3-D accelerometer may be passed through one or more bandpass filters 271 to separate the AC-high frequency component. The output of the bandpass filter 271, including primarily only AC-high frequency components, represents an acceleration signature indicative of heart sounds produced during a corresponding cardiac cycle. In one example, the sensor may couple to a bandpass filter 271 for each axis of the accelerometer. In one example, each bandpass filter 271 is the same for each axis, whereas in other examples, each bandpass filter 271 may be different for each axis of the accelerometer. In yet another example, the two bandpass filters may be identical for two axes, and third bandpass filter may be different for a third axis. In an additional or alternative example, in order to detect activity levels, the 3-D accelerometer may include another bandpass filter 271 that may have a different sensitivity setting. In yet another example, each bandpass filter 271 may have two filter settings, with a first filter setting between 7.5-100 Hz and a second filter setting between 15-100 Hz. In such an example, the filter with the 7.5 Hz lower −3 dB is ideal for collecting higher frequency contents, such as provided by a first heart sound S3, whereas the filter with 15 Hz lower −3 dB is a better option in collecting higher frequency content such as a second heart sound S1 or S2 while minimizing low frequency drift, or noise. Therefore, depending on the desired content of the heart sound or frequency characteristics of heart sound or activity level data of interest, a different filter, or setting may be utilized. Specifically, the microcontroller 220 may command the bandpass setting based on operational and patient conditions.

Returning to FIG. 2, a battery 272 provides operating power to all of the components In the IMD 100. The battery 272 is capable of operating at low current drains for long periods of time. The battery 272 also desirably has a predictable discharge characteristic so that elective replacement time may be detected. As one example, the housing 140 employs lithium/silver vanadium oxide batteries. The battery 272 may afford various periods of longevity (e.g., three years or more of device monitoring). In alternate embodiments, the battery 272 could be rechargeable. See, for example, U.S. Pat. No. 7,294,108, titled “Cardiac event micro-recorder and method for implanting same”, which is hereby incorporated by reference.

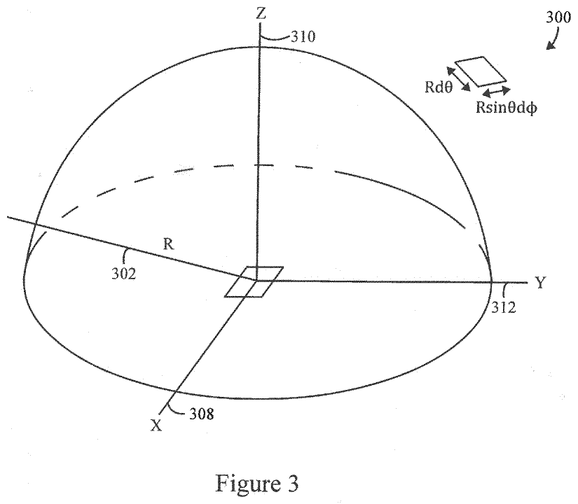

FIG. 3 illustrates force vectors experienced by the IMD 100 as determined by an accelerometer. The microcontroller 220 utilizes device location information, collected from the physiologic sensor 270, or accelerometer, to define a base local device coordinate system 300 for the IMD. The base local device coordinate system 300 may correspond to a global coordinate system and may be defined in terms of various types of coordinate systems, such as a Cartesian coordinate system, Polar coordinate system or otherwise. The microcontroller 220 defines the base local device coordinate system 300 relative to a reference vector 302 that corresponds to and is defined by, the gravitational force of earth. Regardless of the position and orientation of the IMD 100, the gravitational force of earth will remain constant and serve as a reference vector having a fixed magnitude and direction.

After implant, during a calibration procedure, a patient moves through a number of predefined postures that are configured to orient the IMD 100 in known positions and orientations with respect to the gravitational force. When the patient is at each of the predefined postures, the microcontroller 220 collects device location information from the physiologic sensor 270, providing location information in the X, Y and Z directions 308, 312, 310, relative to the Earth's gravitational force. The IMD 100 has an initial/reference position and orientation within the base local device coordinate system 300. For example, the initial/reference position and orientation may define an orientation of a longitudinal axis extending through a center of the IMD 100 and may define a position of a reference point on the IMD 100 (e.g., a distal or proximal tip, a center of mass, a center point on a select electrode and the like). After the calibration procedure, the calibration acceleration signatures are utilized to determine an axis of the accelerometer associated with a current posture. The confirmation acceleration signatures are obtained along the axis of the accelerometer in connection with the analyzing the far field CA signals.

### Methods for Labeling Arrythmias

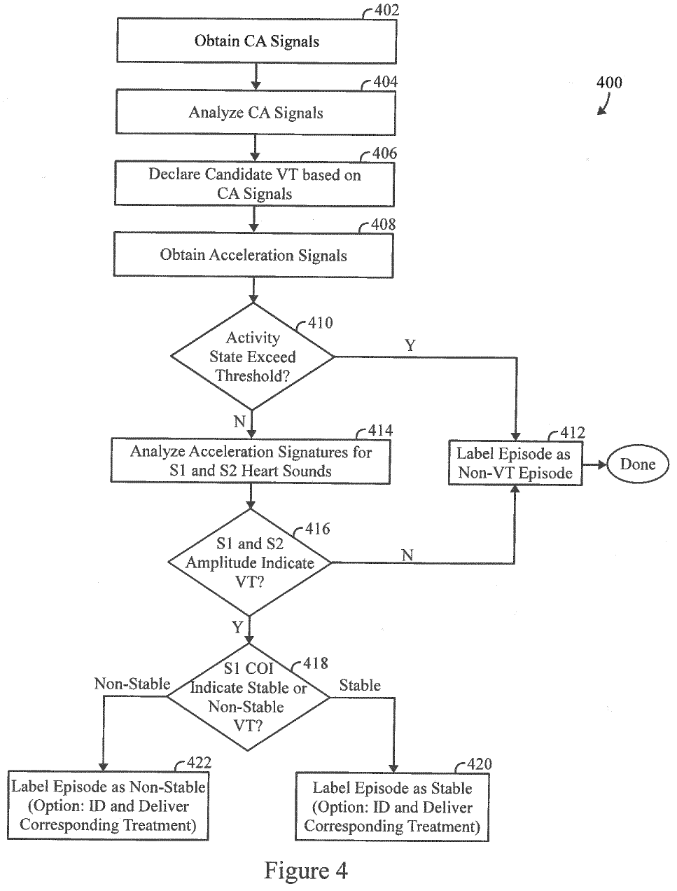

FIG. 4 illustrates a computer-implemented method 400 for labeling types of heart arrhythmias based on cardiac activity. In one example, the method 400 is performed utilizing the CA signals and accelerometer signatures detected and retrieved by the systems and methods described in detail in relation to FIGS. 1-3. All or a portion of the operations of FIG. 4 may be implemented by one or more processors of the IMD configured with executable instructions. It should be recognized that while the operations of method 400 are described in a somewhat serial manner, one or more of the operations of method 400 may be continuous and/or performed in parallel with one another. For example, the various operations of the IMD 100 may be continuous and/or performed in parallel with one another and/or other functions of the IMD. Also, unless otherwise indicated, each operation of method 400 is performed under the control of one or more processors configured with program instructions.

Beginning at 402, the one or more processors of the IMD obtain CA signals at electrodes (e.g., one or more of electrodes 126, 127, 128) during cardiac beats.

At 404, the one or more processors of the IMD analyze the CA signals to assess the presence or absence of a candidate arrhythmia episode (e.g., a candidate VT episode). In one example, the arrhythmia detection circuitry 234 of FIG. 2 analyzes the CA signals to assess the rate of cardiac beats, the morphology of the QRS complexes, the duration of the QRS complexes, the AR intervals, T-wave morphology, and the like. Upon detection of a candidate arrhythmia episode, the arrhythmia detection circuitry 234 transmits a signal to the one or more processors indicating that a candidate arrhythmia episode (e.g., a candidate VT episode) is presented. Optionally, the one or more processors record an ensemble of cardiac beats and utilize a mathematical operation (e.g., averaging, mean, median, and the like) to combine the CA signals to form resultant CA signals that are analyzed for the candidate arrhythmia.

At 406, the one or more processors of the IMD declare a candidate VT episode based on the CA signals. The VT episode is declared to represent a “candidate” as embodiments herein implement further operations to label the type of VT episode and/or confirm that a VT episode is in fact occurring. For example, embodiments herein may, after further analysis, deny the candidate VT episode due to various factors such as T-wave oversensing and the like. Optionally, embodiments herein do not implement any further “confirmation” of the candidate VT episode, but instead limit the further analysis to merely labeling the type of VT episode as stable or non-stable, in which case the VT episode is considered a “candidate” to the extent the particular type is not yet known at the time of 406.

At 408, the one or more processors of the IMD obtain acceleration signatures at a physiological sensor of the IMD that are indicative of activity level and heart sounds generated during the cardiac beats. In one example, the physiological sensor is physiological sensor 270 of FIG. 2 and is an accelerometer. In the example, the one or more processors obtain the acceleration signatures from or about one or more axes, X, Y, or Z, of the accelerometer. The acceleration signatures may be for the same series of cardiac beats as the CA signals. Additionally, or alternatively, the acceleration signatures may be for a series of cardiac beats following the cardiac beats for which the CA signals were obtained. Optionally, the acceleration signatures may be for a series of cardiac beats that include at least some cardiac beats of the series of cardiac beats as the CA signals and cardiac beats following the same series of cardiac beats as the CA signals.

At 410, the one or more processors of the IMD evaluate the acceleration signatures to confirm or deny the candidate arrhythmia episode (e.g., the candidate VT episode). In one example, the acceleration signatures are analyzed for an activity level experienced by the patient. The activity level may be a stationary state, a rest state, an exercise state, a walking state, and the like. In the example, the process confirms or denies the candidate VT episode based on a relation between the activity level and a threshold. Accordingly, comparing an activity level to a threshold may indicate if a patient is moving versus stationary (e.g., unconscious) during and/or after the time the CA signals are collected. If the activity level exceeds the threshold, the process interprets the condition to deny the candidate VT episode, indicating a non-VT episode, and flow moves to 412. For example, if the activity level exceeds a threshold, the activity level may indicate that the patient is conscious. The patient may remain conscious when the patient is able to tolerate a true arrhythmia episode well. Optionally, the activity level may exceed the threshold when a false arrhythmia detection occurs, such as based on over sensing of T-waves. Thus, when the activity level exceeds the threshold, the process determines that it is appropriate to deny the candidate VT episode.

If the activity level does not exceed the threshold, the process interprets the condition to confirm the candidate VT episode, and flow moves to 414. For example, if the activity level corresponding to a candidate VT episode falls below a threshold, the activity level indicates that the patient is unconscious or exhibits rapidly decreasing activity, confirming the candidate VT episode.

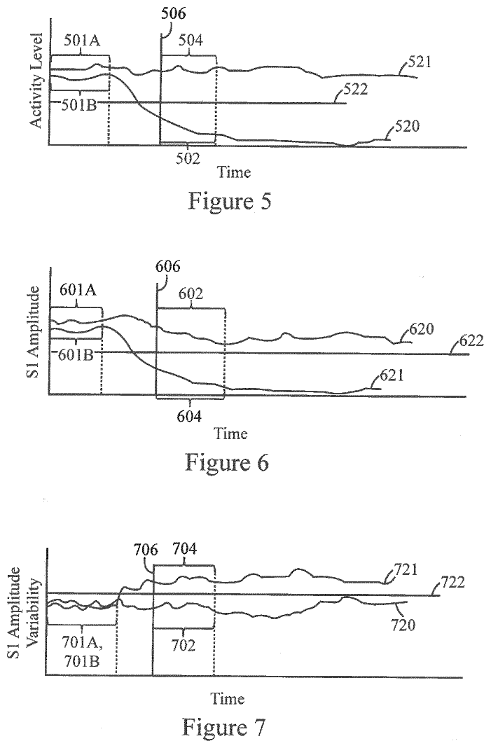

FIG. 5 is a graph illustrating examples of activity levels exhibited in connection with different types of cardiac activity. The horizontal axis represents time and the vertical axis represents a level of activity. Graph 520 corresponds to a confirmed VT episode. Graph 521 corresponds to a denied VT episode. Graph 522 corresponds to an activity level threshold below which a patient is deemed inactive, and above which a patient is deemed active. Graph 520 illustrates one example of a confirmed VT episode, where a patient exhibits normal activity 501A (e.g., while the patient is walking or otherwise moving) prior to the VT episode detected at 506. When the patient experiences a VT episode (as detected at 506), the patient may fall, sit, or lay down and will become unconscious. When unconscious, the patient's activity level drops to at or near zero as indicated by the VT-related activity level at 502, which is below the activity level threshold illustrated by graph 522. Graph 521 illustrates one example of a denied VT episode, where a patient exhibits normal activity 501B (e.g., while the patient is walking or otherwise moving) both prior to the VT episode detected at 506 and thereafter. When the one or more processors of the IMD detect an arrhythmia event (as detected at 506), and the patient's activity level remains normal as indicated by the VT-related activity level at 504, which is above the activity level threshold illustrated by graph 522, the process 400 denies the VT episode.

With respect to the operations of FIG. 4, the one or more processors of the IMD obtain (at the onset of detection 506 and for the select number of cardiac beats) and analyze CA signals at 402, 404, and based thereon, declare a candidate VT episode 502, 504 at operation 406. The one or more processors obtain acceleration signatures associated with the respective candidate VT episode 502, 504, indicative of activity level and heart sounds, at operation 408. At 410, the one or more processors compare an activity level of the candidate VT episode 502, 504 with a threshold 522. If the one or more processors determine the activity level of the candidate VT episode 504 exceeds the threshold 522 at the time of detection 506, the process interprets the condition to indicate a non-VT episode, denying the candidate VT episode. If the one or more processors determine the activity level of the candidate VT episode to fall below the threshold 522 at the time of detection 506, the process interprets the condition to confirm the candidate VT episode based on the activity level.

At 412, when the process denies the candidate VT episode based on the activity level, the one or more processors of the IMD label the candidate VT episode as a non-VT episode and the process ends. One example of a treatment administered by an IMD for a VT episode may include delivering an electrical stimulation therapy such as a defibrillation therapy or the like. Delivery of an electrical stimulation therapy by the IMD while the patient is conscious, either because the candidate VT episode is well-tolerated by the patient or the candidate VT episode is inappropriate due to sensing issues or hardware dysfunctions, may adversely impact the quality of life of the patient.

At 414, the one or more processors of the IMD analyze the acceleration signatures for S1 and S2 HS.

At 416, the one or more processors of the IMD analyze the S1 and S2 HS components of the acceleration signatures to confirm or deny the candidate VT episode. In a VT episode, left ventricular (LV) function worsens, leading to a reduction in pressure. The amplitudes of the S1 and S2 HS components are linked to LV function and pressure, and may be compared to one or more of a predetermined fixed threshold, a dynamic threshold, S1 and S2 amplitudes acquired previously during exercise. S1 and S2 thresholds obtained during a VT/ventricular fibrillation (VF) induction test, or the like to confirm or deny candidate VT episodes. Accordingly, analyzing the S1 and S2 components may include comparing the amplitudes of the S1 and S2 HS components to a predetermined fixed threshold. Analyzing the S1 and S2 components may also include comparing the amplitudes of the S1 and S2 HS components to a dynamic threshold that is derived based on S1 and S2 trends over a previous pre-determined window of the cardiac beats after determination of a candidate VT episode. A dynamic threshold may be derived based on the one or more processors recording an ensemble of amplitude values for the S1 and S2 components over the pre-determined window of cardiac beats and utilizing a mathematical operation (e.g., averaging, mean, median, and the like) to combine the ensemble of amplitude values for the S1 and s2 components, respectively, to derive the dynamic threshold. Analyzing the S1 and S2 components may also include comparing the amplitudes of the S1 and S2 HS components to S1 and S2 amplitudes that were acquired previously during a VT/VF induction test. Analyzing the S1 and S2 components may also include comparing the amplitudes of the S1 and S2 HS components to S1 and S2 amplitudes that were acquired previously during an exercise activity and while a heart rate was in a range corresponding to a current heart rate associated with the VT episode. At similar high heart rates, S1 and S2 amplitudes are much higher during exercise versus at rest. Thus, the S and S2 amplitudes corresponding to exercised-induced elevated hear rates may serve as a threshold (or a reference). Based on whether the S1 and S2 amplitude of a candidate VT episode is above or below the corresponding threshold, a VT episode may be confirmed or denied.

Additionally, or alternatively, at 416, the one or more processors of the IMD analyze the amplitude of the S1 HS component to confirm or deny the candidate VT episode. Analyzing the amplitude of the S1 HS component may include one or more of (i) comparing the amplitudes of the S1 HS component to a predetermined threshold, (ii) comparing the amplitude of the S1 HS component to a dynamic threshold that is derived based on S1 trends over a previous window of the cardiac beats, (iii) comparing the amplitudes of the S1 HS component to S1 amplitudes that were acquired previously during a VT/VF induction test, and (iv) comparing the amplitude of the S1 HS component to S1 amplitudes that were acquired previously during an exercise activity and while a heart rate was in a range corresponding to a current heart rate associated with the VT episode similar to the above.

At 416, if the one or more processors determine that the amplitudes of the S1 and S2 HS components exceed a corresponding threshold, the process interprets the condition to deny the candidate VT episode, indicating a non-VT episode, and flow returns to 412. Additionally, or alternatively, if the one or more processors determine that the amplitude of the S1 HS component exceeds the corresponding threshold, the process interprets the condition to deny the candidate VT episode, indicating a non-VT episode, and flow returns to 412. At 412, when the process denies the candidate VT episode based on the amplitude of the S1 and S2 HS components, and/or the amplitude of the S1 component, the one or more processors of the IMD label the candidate VT episode as a non-VT episode and the process ends.

Alternatively, at 416, if the one or more processors determine that the amplitudes of the S1 and S2 HS components do not exceed (or do fall below) a corresponding threshold, the process interprets the condition to confirm the candidate VT episode based on the amplitude of the S1 and S2 HS components, and flow moves to 418. Additionally, or alternatively, if the one or more processors determine that the amplitude of the S1 HS component does not exceed (or does fall below) a corresponding threshold, the process interprets the condition to confirm the candidate VT episode based on the amplitude of the S1 HS components, and flow moves to 418.

At 418, the one or more processors of the IMD calculate and analyze characteristics of interest (COI) from the acceleration signatures and/or the CA signals to identify the confirmed VT episode as a stable or non-stable VT episode. The COI may be one or more of S1 amplitude, S amplitude variation, a QRS to S1 interval, a QRS complex, and the like. In one example, calculating and analyzing the COI may include tracking a level of one or more COI during or after the VT episode. In another example, calculating and analyzing may include tracking the COI over a series of the cardiac beats. Based on the analyzing, the VT episode is determined to be a stable or non-stable VT episode as described in more detail below.

In one example, the COI is an S1 COI, S1 amplitude. The one or more processors of the IMD track a level of the S1 amplitude during or after the VT episode. In a stable VT episode, the right ventricular (RV) pulse pressure change may not be significantly reduced, whereas the RV pulse pressure reduction may be significant in an unstable VT episode. S1 amplitude, which correlates well with ventricular function, are expected to remain relatively unchanged in a stable VT episode but are significantly reduced in an unstable VT episode. Accordingly, by tracking the level of S1 amplitude, and changes therein, at the time of VT declaration and/or some time thereafter, the VT diagnosis may be verified, and the appropriate therapy administered. If the process determines that the S1 amplitude remains above an amplitude level threshold for a select portion of the VT episode, the one or more processors determines the VT episode to be a stable VT episode, and flow moves to 420. If the process determines that the S amplitude falls below the amplitude level threshold for the select portion of the VT episode, the one or more processors determines the VT episode to be a non-stable VT episode, and flow moves to 422.

FIG. 6 is a graph illustrating examples of S1 amplitude levels exhibited in connection with different types of cardiac activity. The horizontal axis represents time and the vertical axis represents a level of S1 amplitude. Graph 620 corresponds to a stable VT episode. Graph 621 corresponds to an unstable VT episode. Graph 622 corresponds to an S amplitude threshold above which a VT episode is determined to be stable, and below which a VT episode is determined to be unstable. Graph 620 illustrates one example of a stable VT episode, where a patient exhibits normal S1 amplitude 601A prior to the VT episode detected at 606. When the patient experiences a VT episode (as detected at 606), the RV pulse pressure may not be significantly reduced as compared to normal. Accordingly, when S1 amplitude (correlating to ventricular function) remains relatively stable and above an amplitude level threshold (graph 622), as indicated by the stable VT-related S1 amplitude level 602, the process 400 interprets the VT episode to be a stable VT episode. Graph 621 illustrates one example of an unstable VT episode, where a patient exhibits normal S1 amplitude 601B prior to the VT episode detected at 606. When the patient experiences a VT episode (as detected at 606), the RV pulse pressure may be significantly reduced as compared to normal. Accordingly, S1 amplitude is reduced as compared to normal and falls below the amplitude level threshold (graph 622), as indicated by the unstable VT-related S1 amplitude level 604, the process 400 interprets the VT episode to be an unstable VT episode.

In another example, the COI is an S1 COI, S amplitude variation. The one or more processors of the IMD track a level of the S1 amplitude variation during or after the VT episode. S1 amplitude is expected to vary in an unstable VT episode versus a stable VT episode, as the ventricles during an unstable VT episode are subject to greater beat-to-beat variability in ventricular contraction. As such, if the process determines that the S1 amplitude variation is less than an amplitude variation threshold for a select portion of the VT episode, the one or more processors determines the VT episode to be a stable VT episode, and flow moves to 420. If the process determines that the S1 amplitude variation exceeds the amplitude variation threshold for the select portion of the VT episode, the one or more processors determines the VT episode to be a non-stable VT episode, and flow moves to 422.

FIG. 7 is a graph illustrating examples of S1 amplitude variation exhibited in connection with different types of cardiac activity. The horizontal axis represents time and the vertical axis represents S1 amplitude variability. Graph 720 corresponds to a stable VT episode. Graph 721 corresponds to an unstable VT episode. Graph 722 corresponds to an S amplitude variation threshold below which a VT episode is determined to be stable, and above which a VT episode is determined to be unstable. Graph 720 illustrates one example of a stable VT episode, where a patient exhibits normal S1 amplitude variation 701A prior to the VT episode detected at 706. When the patient experiences a VT episode (as detected at 706), the RV pulse pressure may not exhibit increased beat-to-beat variability in ventricular contraction as compared to normal. Accordingly, when S1 amplitude variation (correlating to ventricular function) remains relatively stable and falls below an amplitude variability threshold (graph 722), as indicated by the stable VT-related S1 amplitude level 702, the process 400 interprets the VT episode to be a stable VT episode. Graph 721 illustrates one example of an unstable VT episode, where a patient exhibits normal S1 amplitude variation 701B prior to the VT episode detected at 706. When the patient experiences a VT episode (as detected at 706), the RV pulse pressure may be significantly reduced as compared to normal. Accordingly, when S1 amplitude variation is increased as compared to normal and exceeds the amplitude level threshold (graph 722), as indicated by the unstable VT-related S1 amplitude level 704, the process 400 interprets the VT episode to be an unstable VT episode.

In another example, the COI is an S1 COI and includes S1 amplitude level and variation. The one or more processors of the IMD track a level and variation of the S1 amplitude during or after the VT episode. The combination of S1 amplitude level and variation may lead to improved differentiation over each parameter alone. If the process determines that the level and variation of the S1 amplitude has a first relationship to the variation and amplitude thresholds for a select portion of the VT episode, the one or more processors determines the VT episode to be a stable VT episode, and flow moves to 420. If the process determines that the level and variation of the S1 amplitude has a second relationship to the variation and amplitude thresholds for a select portion of the VT episode, the one or more processors determines the VT episode to be a stable VT episode, and flow moves to 422.

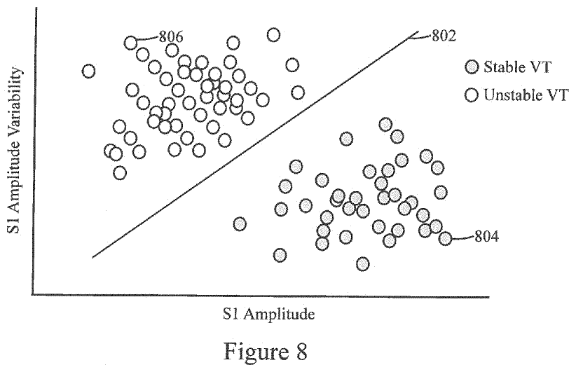

FIG. 8 illustrates a graph of S1 amplitude variability versus S1 amplitude level that may result from the analysis undertaken at 418. As illustrated, the black line represents the threshold line 802 that separates a stable VT episode and an unstable VT episode. In one example of 418, when both the S1 amplitude variability and the S1 amplitude level fall to the right side 804 of the threshold line 802 (e.g., a first relationship), the process determines the VT episode to be a stable VT episode. In another example of 418, when both the S1 amplitude variability and the S1 amplitude level fall to the left side 806 of the threshold line 802 (e.g., a second relationship), the process determines the VT episode to be an unstable VT episode.

In another example, the COI is a QRS to S1 interval. The QRS to S1 interval in a stable VT episode may remain within a selected threshold while the QRS to S1 interval In an unstable VT episode exceeds the selected threshold. Accordingly, the QRS to S1 variability may differentiate between a stable and an unstable VT episode. As such, the one or more processors of the IMD identify, over a series of the cardiac beats, a QRS to S1 interval corresponding to a period of time between i) a feature of interest In a QRS complex of the corresponding cardiac beats and ii) a feature of interest in the S1 heart sound. If the process determines that the QRS to S1 variability over the series of cardiac beats is less than a QRS to S1 threshold, the one or more processors determines the VT episode to be a stable VT episode, and flow moves to 420. If the process determines that the QRS to S1 variability over the series of cardiac beats exceeds the QRS to S1 variability threshold, the one or more processors determines the VT episode to be a non-stable VT episode, and flow moves to 422.

In yet another example, the COI includes a QRS complex. The one or more processors of the IMD identify, over a series of the cardiac beats, a QRS complex from the corresponding cardiac beats, and detect skipped S1 heart sounds that do not occur within an S1.search window following a corresponding QRS complex. In order to detect skipped S1 heart sounds, the one or more processors detect S1 heart sounds in an S search window following a QRS complex and track the number times an S1 heart sound does not occur in the S1 search window following each QRS detection over a series of cardiac beats. In a stable VT episode, the QRS and S1 association is expected to be maintained, whereas S1 may not occur in some QRS detections for an unstable VT detection. If the process determines that number of skipped S1 heart sounds is maintained within a select range, the process determines the VT episode to correspond to a stable VT episode and flow moves to 420, over the series of cardiac beats is less than a QRS to S1 threshold, the one or more processors determines the VT episode to be a stable VT episode, and flow moves to 420. If the process determines that number of skipped S1 heart sounds is outside of a select range, the process determines the VT episode to correspond to an unstable VT episode and flow moves to 422.

At 420, the one or more processors of the IMD label the VT episode to be a stable VT episode. Optionally, based on determining a stable VT episode, the one or more processors may deliver a first therapy for treating a stable VT episode.

At 422, the one or more processors of the IMD label the VT episode to be an unstable VT episode. Optionally, based on determining an unstable VT episode, the one or more processors may deliver a second therapy for treating an unstable VT episode.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It Is to be understood that the subject matter described herein is not limited In its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A computer implemented method for labeling types of heart arrhythmias based on cardiac activity, comprising:
under control of one or more processors of an implantable medical device (IMD) configured with specific executable instructions;
obtaining cardiac activity (CA) signals at electrodes of the IMD during cardiac beats;
declaring a ventricular tachycardia (VT) episode based on the CA signals;
obtaining acceleration signatures, at an accelerometer of the IMD, indicative of heart sounds generated during the cardiac beats;
analyzing an S1 characteristic of interest (COI) from the acceleration signature to identify the VT episode as a stable or non-stable VT episode; and
labeling the VT episode as stable or non-stable based on the analyzing operation.

2. The method of claim 1, wherein the declaring includes declaring the VT episode as a candidate VT episode, the method further comprising analyzing S1 and S2 heart sound (HS) components of the acceleration signatures to confirm or deny the candidate VT episode.

3. The method of claim 2, wherein the analyzing the S1 and S2 HS components comprises at least one of:
i) comparing amplitudes of the S1 and S2 HS components to a predetermined threshold;
ii) comparing the amplitudes of the S1 and S2 HS components to a dynamic threshold that is derived based on S1 and S2 trends over a previous window of the cardiac beats; and
iii) comparing the amplitudes of the S1 and S2 HS components to S1 and S2 amplitudes that were acquired previously during an exercise activity and while a heart rate was in a range corresponding to a current heart rate associated with the VT episode.

4. The method of claim 1, wherein the declaring includes declaring the VT episode as a candidate VT episode, and wherein the analyzing further comprises analyzing an amplitude of an S1 heart sound component to confirm or deny the candidate VT episode.

5. The method of claim 1, wherein the S1 COI is S1 amplitude and the analyzing further comprises:
tracking a level of the S1 amplitude during or after the VT episode;
labeling the VT episode as stable when the level of the S1 amplitude remains above an amplitude level threshold for a select portion of the VT episode; and
labeling the VT episode as non-stable when the level in the S1 amplitude falls below the amplitude level threshold for the select portion of the VT episode.

6. The method of claim 1, wherein the S1 COI is S1 amplitude variation and the analyzing further comprises:
tracking variation in the S1 amplitude during or after the VT episode;
labeling the VT episode as stable when the variation of the S1 amplitude is less than an amplitude variation threshold for a select portion of the VT episode; and
labeling the VT episode as non-stable when the variation in the S1 amplitude exceeds the amplitude variation threshold for the select portion of the VT episode.

7. The method of claim 1, wherein the S1 COI is S amplitude level and variation and the analyzing further comprises:
tracking variation and a level of the S1 amplitude during or after the VT episode;
labeling the VT episode as stable or non-stable based on a relation between the variation and level of the S1 amplitude and variation and amplitude thresholds.

8. The method of claim 1, further comprising:
identifying, over a series of the cardiac beats, a QRS to S1 interval corresponding to a period of time between i) a feature of interest in a QRS complex of the corresponding cardiac beats and ii) a feature of interest in the S1 heart sound; and
determining a variability of the QRS to S1 interval over the series of the cardiac beats;
wherein the labeling further comprising labeling the VT episode as stable or non-stable based on the variability of the QRS to S1 interval.

9. The method of claim 1, further comprising:
identifying, over a series of the cardiac beats, a QRS complex from the corresponding cardiac beats;
detecting skipped S1 heart sounds that do not occur within an S1 search window following a corresponding QRS complex; and
wherein the labeling further comprising labeling the VT episode as stable or non-stable based on the skipped S1 heart sounds.

10. The method of claim 1, further comprising analyzing the acceleration signature for an activity level; and confirming or denying the VT episode based on a relation between the activity level and a threshold, wherein the activity level is indicative of a non-VT episode when the activity level exceeds the threshold, wherein the activity level is indicative of the VT episode when the activity level falls below the threshold.

11. The method of claim 1, further comprising delivering a first therapy when the VT episode is labeled as stable and delivering a second therapy when the VT episode is labeled as non-stable.

12. A system for labeling types of heart arrhythmias based on cardiac activity, the system comprising:
one or more processors;
and a memory coupled to the one or more processors, wherein the memory stores program instructions, wherein the program instructions are executable by the one or more processors to:
obtain cardiac activity (CA) signals at electrodes of the IMD during cardiac beats;
declare a ventricular tachycardia (VT) episode based on the CA signals;
obtain acceleration signatures, at an accelerometer of the IMD, indicative of heart sounds generated during the cardiac beats;
analyze an S1 characteristic of interest (COI) from the acceleration signature to identify the VT episode as a stable or non-stable VT episode; and
label the VT episode as stable or non-stable based on the analyzing operation.

13. The system of claim 12, wherein the declare includes declare the VT episode as a candidate VT episode, wherein the program instructions are further executable by the one or more processors to analyze S1 and S2 HS components of the acceleration signatures to confirm or deny the candidate VT episode.

14. The system of claim 13, wherein the analyze S1 and S2 HS components comprises at least one of:
i) compare amplitudes of the S1 and S2 heart sound (HS) components to a predetermined threshold;
ii) compare the amplitudes of the S1 and S2 HS components to a dynamic threshold that is derived based on S1 and S2 trends over a previous window of the cardiac beats; and
iii) compare the amplitudes of the S1 and S2 HS components to S1 and S2 amplitudes that were acquired previously during an exercise activity and while a heart rate was in a range corresponding to a current heart rate associated with the VT episode.

15. The system of claim 12, wherein the declaring includes declaring the VT episode as a candidate VT episode, and wherein the analyzing further comprises analyzing an amplitude of an S1 HS component to confirm or deny the candidate VT episode.

16. The system of claim 12, wherein the S1 COI is S1 amplitude and the analyze further comprises:
track a level of the S1 amplitude during or after the VT episode;
label the VT episode as stable when the level of the S amplitude remains above an amplitude level threshold for a select portion of the VT episode; and
label the VT episode as non-stable when the level in the S1 amplitude falls below the amplitude level threshold for the select portion of the VT episode.

17. The system of claim 12, wherein the S1 COI is S1 amplitude variation and the analyzing further comprises:
track variation in the S1 amplitude during or after the VT episode;
label the VT episode as stable when the variation of the S1 amplitude is less than an amplitude variation threshold for a select portion of the VT episode; and
label the VT episode as non-stable when the variation in the S1 amplitude exceeds the amplitude variation threshold for the select portion of the VT episode.

18. The system of claim 12, wherein the S1 COI is S amplitude level and variation and the analyze further comprises:
track variation and a level of the S1 amplitude during or after the VT episode;
label the VT episode as stable or non-stable based on a relation between the variation and level of the S1 amplitude and variation and amplitude thresholds.

19. The system of claim 12, wherein the program instructions are further executable by the one or more processors to:
identify, over a series of the cardiac beats, a QRS to S1 interval corresponding to a period of time between i) a feature of interest in a QRS complex of the corresponding cardiac beats and ii) a feature of interest in the S1 heart sound; and
determine a variability of the QRS to S1 interval over the series of the cardiac beats;
wherein the label further comprises label the VT episode as stable or non-stable based on the variability of the QRS to S1 interval.

20. The system of claim 12, wherein the program instructions are further executable by the one or more processors to:
identify, over a series of the cardiac beats, a QRS complex from the corresponding cardiac beats;
detect skipped S1 heart sounds that do not occur within an S1 search window following a corresponding QRS complex; and
wherein the label further comprises label the VT episode as stable or non-stable based on the skipped S1 heart sounds.
